(19) United States
(12) Patent Application Publication
(10) Pub. No.: US 2011/0136727 A1
(43) Pub. Date: Jun. 9, 2011

(54) Compositions and methods for rapid selection of pathogen binding agents

(76) Inventor: SERGEI SVAROVSKY, SAN DIEGO, CA (US)

(21) Appl. No.: 12/951,034

(22) Filed: Nov. 20, 2010

Related U.S. Application Data
(60) Provisional application No. 61/263,171, filed on Nov. 20, 2009.

(51) Int. Cl. A61K 38/02; C07K 2/00; C07K 9/00; C40B 40/10; A01N 37/18; C40B 30/04; A01P 1/00; A61P 31/04

(52) U.S. Cl. 514/2.4; 530/300; 525/54.1; 530/322; 506/18; 506/9

(57) ABSTRACT

Isolated glycan binding peptide complex comprise two or more glycan binding peptides operatively coupled to each other. These are bacterial binding peptide conjugates (e.g., glycan binding peptides) to a multivalent polymer (e.g., a multivalent PEG molecule) or to the surface of particles that create multimeric constructs that inhibit growth and aggregation of microbes. Included is a method of evaluating a substance for the presence of a microbe comprising contacting the substance with a peptide microarray or a peptide complex comprising a plurality glycan binding peptide operatively coupled to a substrate or multivalent linker, wherein the glycan binding peptide is coupled to an array by a linker that is at least 0.5 micrometers in length.

### CROSS-REFERENCE TO RELATED APPLICATION

This application claims the benefit of U.S. Provisional Application No. 61/263,171, filed 20 Nov. 2009, which is hereby incorporated by reference herein

### TECHNICAL FIELD

Embodiments of this invention are directed generally to biology and medicine. Certain aspects are directed to compositions comprising glycan binding peptide complexes and uses thereof.

### BACKGROUND ART

Bacterial resistance to traditional antibiotics has reached alarming levels, thus there is a strong need to develop new antimicrobial agents with novel modes of action and/or different cellular targets compared to the existing antibiotics. While most antibiotics act on enzymes involved in cell wall synthesis, agents that target membrane glycans directly have not been fully explored in a clinical setting—particularly agents with selective specificity for bacterial glycans. In previous studies aptamers (small synthetic pieces of DNA) have been able to inhibit pathogenic invasion by sticking to the surface of pathogens.

Gene-encoded antimicrobial peptides are ubiquitous components of host defense systems in animals and plants. Accumulated evidence suggests that most of these peptides act by first attaching to the pathogenic glycans, e.g., lipopolysaccharides (LPS/endotoxins) in gram-negative bacteria, and then permeabilizing the cell membranes of the microorganisms and killing them.

The increasing development of bacterial resistance to traditional antibiotics has created a strong need to develop new antimicrobial agents with novel modes of action and/or different cellular targets compared to the existing antibiotics.

### SUMMARY OF INVENTION

Many pathogens have large quantities of glycans on their cell surface in the form of polysaccharides, peptidoglycans, and other species-specific biopolymers (such as lipoteichoic acid in gram positive bacteria). In medical situations, these surface components of bacterial cells are major determinants of virulence for most pathogens including viruses, bacteria, fungi, and parasites. The term “glycan” refers to the carbohydrate portion of a glycoconjugate, such as a glycopeptide, glycoprotein, glycolipid, or proteoglycan. Generally, glycans tend be oligosaccharides or polysaccharides. The glycan-binding peptide is a peptide or peptide-like molecule that binds glycans. The term “peptide” includes peptides, polypeptides, proteins and fragments of proteins, peptoids, peptidomimetics, and peptide-like molecules that contain non-naturally occurring amino acids, peptoids and the like. Glycan binding peptides can be identified using screening procedures. When microbial binding is conducted in the presence of excess glycan those peptides whose binding is inhibited by the excess glycan are specific for binding to bacteria having that particular glycan on its surface.

Certain embodiments of the invention include conjugation of bacterial binding peptides (e.g., glycan binding peptides) to a multivalent polymer (e.g., a multivalent PEG molecule) or to the surface of particles creating multimeric constructs. These multimeric constructs enhance the peptides capability to inhibit growth and aggregate microbes. In certain aspects, LPS binding peptide motifs can be presented on a suitable multivalent scaffold to enhance bacterial binding. In further aspects, composition of the current application can be used, but are not limited to (1) antibacterial/antiseptic shock therapeutics; (2) antimicrobial surface coatings; (3) pathogen detection; (4) magnetic separation; and/or (5) endotoxin purification.

Certain embodiments are directed to a high throughput process to select specific bacterial cell surface component binding peptides and methods to prepare multimeric constructs of such peptides that significantly enhance their ability to interact with bacterial cells. By specifically adhering to the pathogen's surface, these synthetic constructs may not only help incapacitate or kill the microorganism but prevent the invasion of host cells and expose the pathogen to the host immune system for elimination. Since cell surface components are often vital for the pathogen's survival, the genetic barrier to escape the pressure is high, thus precluding the development of resistance. These multiple modi operandi are expected to prevent easy selection of mutants and work against clinically common drug-resistant microorganisms in general.

Certain embodiments include an isolated glycan binding peptide complex comprising 2, 3, 4, 5, 6, 7, 8, 9, 10, 50, or more glycan binding peptides, including all values and ranges there between, operatively coupled to each other. In certain aspects, the glycan binding peptide complex comprises at least 4 glycan binding peptides. In further aspects, the glycan binding peptide complex comprises at least 10 glycan binding peptides. The glycan binding peptides can be coupled via a linker moiety. In certain aspects, the linker moiety is a polymer. In further aspects, the polymer is a polyethylene glycol polymer or polyethyleneimine polymer. In the case of multimeric peptide complex the linker is a multivalent linker comprising a plurality of groups for attachment of the peptide to the linker. In certain aspects, a multivalent polymer that can be coupled to at least 3, 4, 5, 6, 7, 8, 9, 10, or more glycan binding peptides. The glycan binding peptide complex can comprise glycan binding peptides that have the same, similar, or different amino acid sequences. In certain aspects, the glycan binding peptides are coupled to a particle. In certain aspects, the particle is a magnetic particle.

Further embodiments include peptide microarrays comprising a plurality of glycan binding peptides each operatively coupled to a substrate by a linker moiety that is at least, about, or at most 0.5, 1, 5, 10, 20, 30, 40, 50, 100 micrometers or more in length. In a further aspect, a linker may include 5, 10, 20, 50, 100, 500, 1,000 or more repeating units or monomer units, including all values and ranges there between. In certain aspects the linker moiety is a water soluble branched or linear, rigid, semi-rigid, or flexible polymer, such as polyethylene glycol (PEG), polyethyleneimine (PEI), polyvinyl alcohol (PVA), polyacrylamide (PLA), polylactic acid (PL), polyglycolide (PG), and poy(lactic-co-glycolic) acid (PLGA) together with their copolymers with either hydrophobic or hydrophilic monomers, poly(glycine), poly(proline), and various amino acid and nucleic acid polymers. In certain aspects, an array substrate is a bead or a planar surface.

Certain embodiments include methods of attenuating microbial growth or infection or invasion comprising contacting bacteria with a glycan binding peptide complex described herein. In certain aspects, the microbes are on a surface or in a solution. In further embodiments the microbes are in a subject or in the tissues or biologic fluids of a subject infected with a microbe. Biologic fluids include blood, urine, sputum, semen, and the like.

Further embodiments include methods of evaluating or isolating or removing a microbe from a substance comprising: (a) contacting a substance (natural, artificial, or biological) with a peptide microarray or a peptide complex comprising a plurality of glycan binding peptides operatively coupled to a substrate as described herein. In certain aspects, the substance or subject is also administered or contacted with a second antimicrobial therapy or agent.

Other embodiments of the invention are discussed throughout this application. Any embodiment discussed with respect to one aspect of the invention applies to other aspects of the invention as well and vice versa. The embodiments in the Example section are understood to be embodiments of the invention and may be applicable to other aspects of the invention.

The terms “inhibiting,” “reducing,” or “prevention,” or any variation of these terms, when used in the claims and/or the specification includes any measurable decrease or complete inhibition to achieve a desired result. In certain aspects the desired result is the decrease or attenuation of microbial invasion, growth, or infection.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the invention, and vice versa. Furthermore, compositions and kits of the invention can be used to achieve methods of the invention.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.” It is also contemplated that anything listed using the term “or” may also be specifically excluded.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Synthetic scheme for preparation of microarray on slides: (a) 1% (w/v) ₂HN-PEG3,400-NH₂ in 0.1M NaHCO₃ at pH9, 30 minutes; (b) sulfo-SMCC, 1×PBS at pH 7.4, 30 minutes; (c) HS-peptide in 1×PBS at pH 7.4, 12 hours.

FIG. 2. Schematic representation of the bacterial binding assay. PEG3,400 linker modified random sequence peptide microarrays were incubated (FIG. 2A) with fluorescently labeled bacteria and (FIG. 2B) with bacteria in presence of excess free LPS. Peptide spots that were quenched or significantly diminished by added LPS were selected as LPS-binding peptides. Insert in FIG. 1A shows a microscope image of fluorescently labeled bacteria binding to one of the array spots.

FIG. 3. Heat map compares the levels of raw luminescent intensity (log 2) between direct E. coli O111:B4 (ECO) binding and binding of ECO in presence of excess LPS (ECO+LPS). In this image, the range of fluorescent intensity on the microarray is represented by red (highest) to blue (lowest). Only top 32 peptides with 2.8 to 18.7 ECO/(ECO+LPS) ratios are shown. Full list and physical properties of 54 peptides with ECO/(ECO+LPS)>2 is available in Table 2.

FIG. 4A Chart of frequencies of amino acids in the sequences of peptide-candidates for binders LPS E. coli O111:B4.

FIG. 4B Distribution of pl for LPS and non-LPS binding peptides.

FIGS. 5A and 5B. Tetravalent (Construct 1) and multivalent (Construct 2) QF8 (SEQ ID NO: 33 Table 2) peptide constructs.

FIG. 6. Bacterial growth inhibition assays in presence of (a) growth media; (b) negative control peptide NEG1 at 100 micromolar; (c) 25 micromolar peptide QF8 (SEQ ID NO: 33 Table 2); (d) 7 micrommolar QF8-PEG Construct 1; (e) 10 micromolar QF8-PEG Construct 1; (f) 17.5 micromolar QF8-PEG construct 1; (g) 25 micromolar QF8-PEG Construct 1; (h) 100 IU penicillin/streptomycin as a positive control.

FIG. 7. Western blot (probed with Streptavidin-AlexaFluor 647) of serum stability of tetravalent Construct 1 (octopus-LPS21 refers to Construct 1 of QF8 (SEQ ID NO: 33 Table 2). Gel shows 2 bands representing both the tetravalent QF8 (upper band) and the isolated QF8 peptide (lower band), which has not been completely removed from the conjugation step. The monomeric peptide (2.75 kDa molecular weight) degraded much faster than that of PEG based tetravalent scaffold, indicating that both PEGylation and oligomerization prolonged the half-life of the conjugate in human serum.

FIG. 8. Adding QF8 (SEQ ID NO: 33 Table 2) functionalized colloidal gold (QF8-AuNP) to E. coli O111:B4 results in nearly instant agglutination of the bacterial cells (insert). TEM image of the granular bacterial precipitate shows QF8-AuNPs (arrows) adhering to the outer lipopolysaccharide layer protruding from the bacterial membrane surface. The scale bar is 0.5 micrometers.

### DESCRIPTION OF EMBODIMENTS

Certain embodiments are directed to additional devices for the identification of glycan binding peptides and/or identification of bacteria that may be present in a sample. Compositions and devices include a plurality of glycan binding peptides operatively coupled to a substrate or surface directly or through a linker. In certain aspects a linker extends the glycan binding peptide above surface to enable interaction with an intact bacterium or microbe. In certain aspects the linker has a length of at least, about, or at most 0.5, 1, 5, 10, 20, 30, 40, 50, 100 micrometers or more. In a further aspect the linker can be rigid, semi-rigid, or flexible. Typically, the linkers will be configured to allow a peptide coupled to the linker to access the surface of a microbe to be bound by the coupled peptide.

The devices and compositions described herein can be used in identifying lead compounds that interact specifically with bacterial cell surface components or can be used as therapeutic agent that bind pathogenic microbes. Typically, the cell surface components are ubiquitously present on the surfaces of most pathogens or are specific for certain pathogens. Uses of the compositions and devices described herein include, but are not limited to use (1) as antimicrobial therapeutics, (2) as antimicrobial coatings, (3) in pathogen detection devices and kits, (4) as decontamination agents or in decontamination kits, and/or (5) as endotoxin purification agents or in endotoxin purification kits.

Antimicrobial Therapeutics. The selected peptides may offer an interesting combination of anti-infective, antibiotic, and immunomodulatory activities that allows alternative design of antibiotics. These materials and concepts provide a new class of antimicrobial compounds to which resistance is not easily acquired. The rapid development of resistance to antibiotics is a major issue for pharmaceutical industry and the development new antibiotics. Certain features of the methods described herein are that they provide rapid access to potential antibiotics that can be quickly developed or evolved into drugable molecules.

Antimicrobial Surface Coatings. With the rapidly proliferating threat of drug-resistant viral and bacterial pathogens there is a great need for coating surfaces of medical devices, toys, household items, door handles, etc. to prevent or reduce risk of infection. Peptides can be easily and cost-effectively produced and immobilized to the surfaces to fulfill this need.

A coating (“coat,” “surface coat,” “surface coating”) includes a liquid, liquefiable or mastic composition that can form a protective or functional surface after application as a thin layer. A surface is the outer layer of any solid object or subject. A coating generally comprises one or more materials that contribute to the properties of the coating, the ability of a coating to be applied to a surface and the ability of the coating to adhere to a surface. Examples of such coating components include a binder, a liquid component, a colorizing agent, an additive, or a combination thereof, and such materials are contemplated for used in a coating.

A coating may be applied to a surface using any technique known in the art. In the context of a coating, “application,” “apply,” or “applying” is the process of transferring of a coating to a surface to produce a layer of coating upon the surface. Application techniques that are contemplated as suitable for a user of little or no particular skill include, for example, dipping, pouring, siphoning, brushing, rolling, padding, ragging, spraying, etc.

Pathogen Detection. Many pathogens express highly characteristic polysaccharides on their surface. In most cases there are no readily available antibodies that can detect these glycan structures. The peptide selected for specific bacterial components can be used to construct pathogen-specific biosensing devices. Such devices will provide pathogen-specific fingerprints or binding characteristics. Economically disadvantaged countries may especially benefit from this low-cost technology that does not require high capital investments in antibody acquisition and complex instrumentation such as PCR.

Cell Isolation and/or Decontamination. The ability to physically separate cells from heterogeneous mixtures can be used for enrichment and purification of target cells, or for the removal of unwanted cells from a substance. Magnetic microbeads have been widely used as a powerful separation tool for cells and bacteria. Compared to flow cytometric cell-sorting, magnetic-based separations offer the clear advantages of rapid, bulk processing of large number of cells. Antibodies form a basis for their use as cell capture and separation tools. One clear disadvantage of using antibodies for cell separation is their cost and bulkiness. Smaller, cheaper molecules would be a welcome addition to the armamentarium of cell separation tools. Short peptides are ideal in this respect as they are easy to synthesize, manipulate, and conjugate to a variety of scaffolds including magnetic beads. Such materials can be used for example as water purification or other bacterial decontamination devices.

Endotoxin purification. Even low levels of endotoxins can be toxic to cells or organisms and must be removed before biological samples can be introduced. Therefore, freeing biological samples from endotoxins is an important biomedical problem. Current methods rely heavily on the use of expensive endotoxins binding proteins and antibodies. Naturally derived and rather expensive cyclic polymyxin B peptide immobilized on beads has also been used for this purpose. Extending these reagents to species-specific peptides may prove beneficial in many situations. The endotoxin-binding peptides identified in this invention can provide a viable low-cost alternative to existing methods.

Glycan Binding Peptides and Compositions

Certain embodiments include peptide arrays and multimeric peptide complexes, and methods of using the same.

Multimeric Complexes

Glycan-binding peptides can be operatively coupled to 2, 3, 4, 5, 6, 7, 8, 9, 10, 50, 100 or more of the same or different glycan binding peptides directly (e.g., to a common multivalent linker) or indirectly (coupled to a common substrate, e.g., a particle). The term “peptide” includes peptides, polypeptides, proteins and fragments of proteins, peptoids, peptidomimetics, and peptide-like molecules that contain non-naturally occurring amino acids, peptoids and the like. In certain aspects, the peptides of a complex are the same. In other aspects, 2, 3, 4, 5, or more different peptides can be coupled to a multimeric linker or a substrate.

The term “linker” means a molecule having at least one end attached or capable of attaching to a solid surface or another molecule and at least one other end having a reactive group that is attached or capable of attaching to a peptide. A linker may already be bound to a solid surface and/or may already have a chemical species of interest bound to its reactive group. A linker may have a protective group attached to its reactive group, where the protective group is chemically or electrochemically removable. A linker may comprise more than one molecule, where the molecules are covalently joined to form the linker having the desired reactive group projecting away from a solid surface. In certain aspects, a linker extends the distance between a peptide and a surface. In certain aspects a linker is multimeric and can be coupled to 3, 4, 5, 6 or more peptides. In a further aspect, 3, 4, 5, 6, 7, 8, 9, 10, or more peptides can be coupled to common support or surface, e.g., a particle. Plurality of linear, cyclic, or branched rigid, semi-rigid, or flexible multimeric scaffolds where multiple chemically reactive sites are expressed as handles for ligand attachment. Examples include, but are not limited to, PAMAM dendrimers, adamantanes, cyclodextrins, calix[n]arenes, multi-armed (2-7) PEGs, polyvalent cyclic and linear peptide and peptoid scaffolds. Two or more different types of molecules can be present simultaneously and in multiple copies on these scaffolds to modulate density and biological activity of the attached ligands.

In certain aspects, a linker can be a polymer. The polymer can be an amino acid polymer. In further aspects, the polymer can be a polyethylene glycol polymer, or polyethyleneimine polymer. “Polyethylene glycol” or “PEG” includes polyalkylene glycol compounds or derivatives thereof, with coupling agents or derivatization with coupling or activating moieties (e.g., with aldehyde, hydroxysuccinimidyl, hydrazide, thiol, triflate, tresylate, azirdine, oxirane, orthopyridyl disulphide, vinylsulfone, iodoacetamide or a maleimide moiety). In accordance with the present invention, useful PEG or other linkers include substantially linear, straight chain, branched, or dendritic molecules. (See, e.g., U.S. Pat. Nos. 5,171,264 and 5,932,462 and 6,602,498).

In other aspects, a linker coupling the peptides of the invention to an array support can be hetero—or homobifunctional linkers. Where necessary, the surface for the array can be derivatized with a bifunctional linker that binds a peptide to the surface. A bifunctional linker generally has a functional group that can covalently binds with a functional group on the surface and a functional group that binds or can be activated to bind a peptide. Examples of homobifunctional linkers include aminoethyl disulfide and heterobifunctional aminopropyl triethoxysilane. In certain embodiments these bifunctional linkers can extend the peptide above surface of the array.

As used herein, the terms “solid support” or “support” refer to any material that provides a solid or semi-solid structure with which another material can be attached. Such materials include smooth supports (e.g., metal, glass, plastic, polycarbonate, silicon, and ceramic surfaces) as well as textured and porous materials. Such materials also include, but are not limited to, gels, rubbers, polymers, dendrimers and other non-rigid materials. Solid supports need not be flat. Supports include any type of shape including spherical shapes (e.g., beads). Materials attached to solid support may be attached to any portion of the solid support (e.g., may be attached to an interior portion of a porous solid support material). Preferred embodiments of the present invention have biological molecules such as proteins attached to solid supports. A biological material is “attached” to a solid support when it is associated with the solid support through a non-random chemical or physical interaction. In some preferred embodiments, the attachment is through a covalent bond. However, attachments need not be covalent or permanent. In some embodiments, materials are attached to a solid support through a “spacer molecule” or “linker group.” Such spacer molecules are molecules that have a first portion that attaches to the biological material and a second portion that attaches to the solid support. Thus, when attached to the solid support, the spacer molecule separates the solid support and the biological materials, but is attached to both. Surfaces and substrates can be coated using a variety of methods including, but not limited to, covalent bond formation via the reaction of surface amino- or carboxyl-groups to peptides or proteins using standard peptide coupling reagents (e.g., 1-ethyl-3-(3-dimethylaminopropyl) carbodiimide hydrochloride).

Arrays

As used herein, the term “microarray” refers to a solid support with a plurality of molecules (e.g., proteins) bound to its surface. Additionally, the term “patterned microarrays” refers to microarray substrates with a plurality of molecules non-randomly bound to its surface.

Peptides and Proteinaceous Compositions

As used herein, a “peptide,” “protein,” or “polypeptide” refers to a molecule comprising at least 5 amino acid residues. In some embodiments, a wild-type version of a peptide, protein or polypeptide are employed, however, in many embodiments of the invention, a modified peptide, protein or polypeptide is employed for glycan binding. The terms described above may be used interchangeably. A “modified peptide” or “modified polypeptide” refers to a peptide or polypeptide whose chemical structure, particularly its amino acid sequence, is altered with respect to a starting peptide. In some embodiments, a modified peptide or polypeptide has at least one modified activity or function such as glycan binding specificity (recognizing that proteins or polypeptides may have multiple activities or functions). It is specifically contemplated that a modified peptide or polypeptide may be altered with respect to one activity or function yet retain another activity or function in other respects.

In certain embodiments the size of a peptide or polypeptide may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 amino molecules or greater, and any range derivable therein, or derivative of a corresponding amino sequence described or referenced herein. It is contemplated that polypeptides may be mutated by truncation, rendering them shorter than their corresponding wild-type form, but also they might be altered by fusing or conjugating a heterologous protein sequence with a particular function (e.g., for targeting or localization, for enhanced immunogenicity, for purification purposes, etc.).

As used herein, an “amino molecule” refers to any amino acid, amino acid derivative, or amino acid mimic known in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

Accordingly, the term “proteinaceous composition” encompasses amino molecule sequences comprising at least one of the 20 common amino acids or at least one modified or unusual amino acid.

Proteinaceous compositions may be made by any technique known to those of skill in the art, including (i) the expression of proteins, polypeptides, or peptides through standard molecular biological techniques, (ii) the isolation of proteinaceous compounds from natural sources, or (iii) the chemical synthesis of proteinaceous materials. The nucleotide as well as the protein, polypeptide, and peptide sequences for various peptide are readily derivable from the amino acid sequence. The coding regions for these peptides may be amplified and/or expressed using the techniques disclosed herein or as would be known to those of ordinary skill in the art.

Amino acid sequence variants of peptides of the invention can be substitutional, insertional, or deletion variants. A modification in a polypeptide of the invention may affect 1, 2, 3, 4, 5, 6, 7, 8, 9, 10, 11, 12, 13, 14, 15, 16, 17, 18, 19, 20 or more non-contiguous or contiguous amino acids of the peptide, as compared to parent or original peptide.

Deletion variants typically lack one or more residues of the parent or original peptide. Individual residues can be deleted or a number of contiguous amino acids can be deleted. A stop codon may be introduced (by substitution or insertion) into an encoding nucleic acid sequence to generate a truncated protein.

Insertional mutants typically involve the addition of material at a non-terminal point in the polypeptide. This may include the insertion of one or more residues. Terminal additions, called fusion proteins, may also be generated.

Substitutional variants typically contain the exchange of one amino acid for another at one or more sites within the peptide, and may be designed to modulate one or more properties of the peptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

The term “functionally equivalent codon” is used herein to refer to codons that encode the same amino acid, such as the six codons for arginine or serine, and also refers to codons that encode biologically equivalent amino acids (see Table 1, below).

TABLE 1
Codon Table

| Amino Acids | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

It also will be understood that amino acid and nucleic acid sequences may include additional residues, such as additional N- or C-terminal amino acids, or 5′ or 3′ sequences, respectively, and yet still be essentially as set forth in one of the sequences disclosed herein, so long as the sequence meets the criteria set forth above, including the maintenance of activity. The addition of terminal sequences particularly applies to nucleic acid sequences that may, for example, include various non-coding sequences flanking either of the 5′ or 3′ portions of the coding region.

The following is a discussion based upon changing of the amino acids of a peptide to create an equivalent, or even an improved, second-generation molecule. For example, certain amino acids may be substituted for other amino acids in a peptide structure without appreciable loss of interactive binding capacity with structures such as, for example, glycans or binding sites on other molecules. Since it is the interactive capacity and nature of a peptide that defines that peptide's function, certain amino acid substitutions can be made in a peptide sequence, and in its underlying DNA coding sequence, and nevertheless produce a peptide with like properties. It is thus contemplated that various changes may be made in the DNA sequence without appreciable loss of their utility or activity of the peptide.

The present invention describes glycan binding polypeptides, peptides, and compositions. In specific embodiments, all or part of the peptides can be synthesized in solution or on a solid support in accordance with conventional techniques. Various automatic synthesizers are commercially available and can be used in accordance with known protocols. Alternatively, recombinant DNA technology may be employed wherein a nucleotide sequence which encodes a peptide of the invention is inserted into an expression vector, transformed or transfected into an appropriate host cell and cultivated under conditions suitable for expression.

Pharmaceutical Compositions and Administration

Combination Therapy

The compositions and related methods of the present invention, particularly administration of a glycan binding peptide complex may also be used in combination with the administration of traditional therapies. These include, but are not limited to, the administration of antibiotics such as streptomycin, ciprofloxacin, doxycycline, gentamycin, chloramphenicol, trimethoprim, sulfamethoxazole, ampicillin, tetracycline or various combinations of antibiotics.

In one aspect, it is contemplated that a peptide and/or therapy is used in conjunction with antibacterial treatment. Alternatively, the therapy may precede or follow the other agent treatment by intervals ranging from minutes to weeks. In embodiments where the other agents and/or peptides are administered separately, one would generally ensure that a significant period of time did not expire between the time of each delivery, such that the agent and peptide composition would still be able to exert an advantageously combined effect on the subject. In such instances, it is contemplated that one may administer both modalities within about 12-24 h of each other and, more preferably, within about 6-12 h of each other. In some situations, it may be desirable to extend the time period for administration significantly, however, where several days (2, 3, 4, 5, 6 or 7) to several weeks (1, 2, 3, 4, 5, 6, 7 or 8) lapse between the respective administrations.

Various combinations may be employed, for example antibiotic therapy is “A” and the glycan binding peptide complex is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the compositions of the present invention to a patient/subject will follow general protocols for the administration of such compounds, taking into account the toxicity, if any, of the glycan binding composition. It is expected that the treatment cycles would be repeated as necessary. It also is contemplated that various standard therapies, such as hydration, may be applied in combination with the described therapy.

General Pharmaceutical Compositions

In some embodiments, pharmaceutical compositions are administered to a subject. Different aspects of the present invention involve administering an effective amount of a composition to a subject. In some embodiments of the present invention compositions may be administered to the patient to protect against infection by one or more microbial pathogens. Additionally, such compounds can be administered in combination with an antibiotic. Such compositions will generally be dissolved or dispersed in a pharmaceutically acceptable carrier or aqueous medium.

The phrases “pharmaceutically acceptable” or “pharmacologically acceptable” refer to molecular entities and compositions that do not produce a significant adverse, allergic, or other untoward reaction when administered to an animal or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in therapeutic compositions is contemplated. Supplementary active ingredients, such as other anti-microbial agents, can also be incorporated into the compositions.

In addition to the compounds formulated for parenteral administration, such as those for intravenous injection, other pharmaceutically acceptable forms include, e.g., tablets or other solids for oral administration; time release capsules; and any other form currently used, including creams, lotions, mouthwashes, inhalants and the like.

The active compounds of the present invention can be formulated for parenteral administration, e.g., formulated for injection via the intravenous, intramuscular, sub-cutaneous, or even intraperitoneal routes. The preparation of an aqueous composition that contains an anti-bacterial compound or compounds will be known to those of skill in the art in light of the present disclosure. Typically, such compositions can be prepared as injectables, either as liquid solutions or suspensions; solid forms suitable for use to prepare solutions or suspensions upon the addition of a liquid prior to injection can also be prepared; and, the preparations can also be emulsified.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization or other sterilization method. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions according to the present invention will typically be via any common route. This includes, but is not limited to oral, nasal, or buccal administration. Alternatively, administration may be by injection. In certain embodiments, a composition may be inhaled (e.g., U.S. Pat. No. 6,651,655, which is specifically incorporated by reference). Such compositions would normally be administered as pharmaceutically acceptable compositions that include physiologically acceptable carriers, buffers or other excipients. As used herein, the term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. The term “pharmaceutically acceptable carrier,” means a pharmaceutically acceptable material, composition or vehicle, such as a liquid or solid filler, diluent, excipient, solvent or encapsulating material, involved in carrying or transporting a chemical agent.

For parenteral administration in an aqueous solution, for example, the solution should be suitably buffered, if necessary, and the liquid diluent first rendered isotonic with sufficient saline or glucose. These particular aqueous solutions are especially suitable for intravenous and intraperitoneal administration. In this connection, sterile aqueous media which can be employed will be known to those of skill in the art in light of the present disclosure. For example, one dosage could be dissolved in isotonic NaCl solution and either added to hypodermoclysis fluid or injected at the proposed site of infusion, (see for example, Remington's Pharmaceutical Sciences, 1990). Some variation in dosage will necessarily occur depending on the condition of the subject. The person responsible for administration will, in any event, determine the appropriate dose for the individual subject.

An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the protection desired.

Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition.

Upon formulation, solutions will be administered in a manner compatible with the dosage formulation and in such amount as is therapeutically or prophylactically effective. The formulations are easily administered in a variety of dosage forms, such as the type of injectable solutions described above.

Kits

Any of the compositions or components described herein may be comprised in a kit. In a non-limiting example, therapeutic reagents; reagents for isolating peptides or complexes thereof, reagents labeling peptides or complexes thereof, and/or reagents and devices for evaluating a sample or composition using an array of glycan binding peptides can be included in a kit, as well reagents for preparation of samples. The kit may further include reagents for creating or synthesizing peptides. The kits can comprise, in suitable container means, a label and/or one or more glycan binding peptides. In certain aspects, the kit can include signal amplification reagents. In other aspects, the kit may include various supports, such as glass, nylon, polymeric beads, magnetic beads, and the like, and/or reagents for coupling any peptide. It may also include one or more buffers, such as reaction buffer, labeling buffer, washing buffer, or a hybridization buffer, and compounds for preparing labeled peptides, microbes, or probes (including agents to compete for peptide binding). Other kits of the invention may include components for making a peptide array comprising linkers of an appropriate length, and thus, may include, for example, a solid support (planar surface or particle).

In specific embodiments, kits of the invention include a peptide array as described in the application. An array may have peptides corresponding to various glycan binding sequences. The subset of peptides on arrays of the invention may be or include those identified as relevant to a particular diagnostic, therapeutic, or prognostic application. For example, the array may contain one or more probes that is indicative or suggestive of (1) a disease or condition, (2) susceptibility or resistance to a particular drug or treatment; and/or (3) diagnostic of development or severity of a disease or condition (prognosis).

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing the nucleic acids, and any other reagent containers in close confinement for commercial sale. Such containers may include injection or blow molded plastic containers into which the desired vials are retained.

However, the components of the kit may be provided as dried powder(s). When reagents and/or components are provided as a dry powder, the powder can be reconstituted by the addition of a suitable solvent. It is envisioned that the solvent may also be provided in another container means.

A kit will also include instructions for employing the kit components as well the use of any other reagent not included in the kit. Instructions may include variations that can be implemented.

Kits of the invention may also include one or more of the following: Control peptides or glycans; protease-free reagents; glycanase-free reagents; and/or protease inhibitors.

It is contemplated that such reagents are embodiments of kits of the invention. Such kits, however, are not limited to the particular items identified above and may include any reagent used for the manipulation or characterization or treatment of microbes.

### EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### Bacterial Growth Inhibition and Agglutinating Activities of Combinatorially Selected Glycospecific Peptides

Microarray Screening. Previously reported was a high-throughput system for selection of LPS binding peptides by using specially designed LPS nanoprobes. Intriguingly most of these peptides showed ability to significantly inhibit bacterial growth. Herein a new and improved system is described that selects peptides that bind to specific bacterial membrane components.

At the core of the selection system is the high-density peptide microarray constructed by spotting 10,410 pre-synthesized 20-mer random sequence cysteine terminated peptides onto an epoxy-functionalized glass microscope slide modified with branched polyethyleneimine (PEI) polymer (MW 25 KDa) or a PEG 3,400 polymer as long linkers terminated with thiol reactive maleimides. In this representation peptides are sufficiently removed from the surface to allow efficient binding to the whole bacteria.

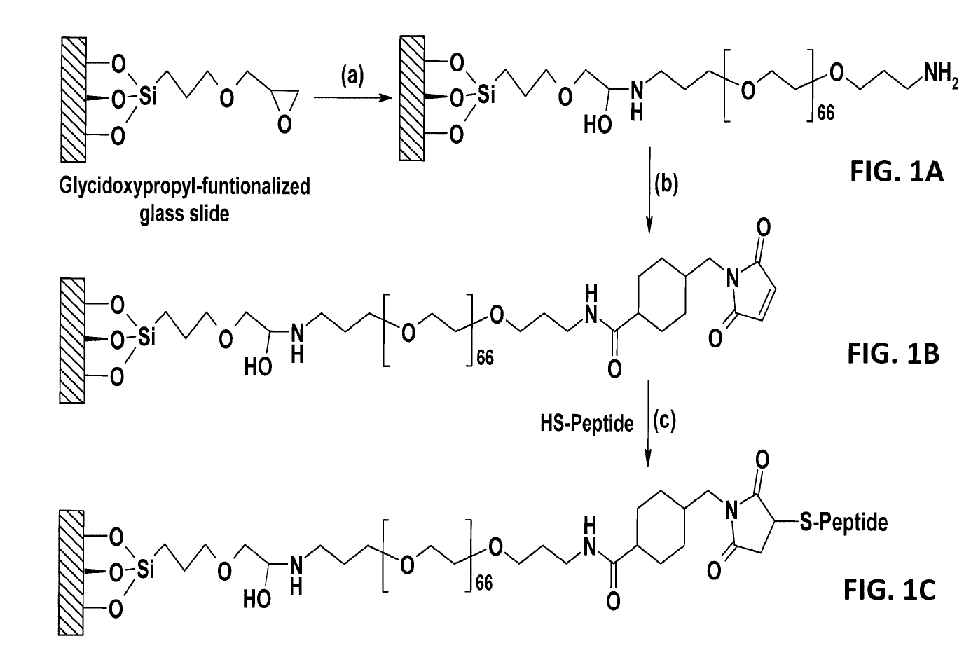

Peptide microarray construction (FIG. 1). High-quality pre-cleaned Gold Seal glass microscope slides were obtained from Fisher. The slides were treated with piranha solution for 30 min, rinsed thoroughly with ddH2O, and spin-dried at 1,000 rpm for 5 min. The slides were then treated with 1% solution of 3-glycidoxypropyltrimethoxysilane in 95% EtOH. The slides were thoroughly washed with absolute ethanol, spin-dried at 1,000 rpm for 5 min, and then cured in convection oven at 150° C. for 1 h. One side of the slides was then reacted with 1% solution of diamine-PEG3,400 (or branched polyethyleneimine, MW 25 KDa) in 0.1M NaHCO3 at pH=9 for 30 min, rinsed well with ddH2O and absolute ethanol and spin-dried at 1,000 rpm for 5 min. Immediately prior to use, the PEG-modified surface was activated for 30 min in 1×PBS at pH=7.4 with the heterobifunctional linker sulfo-SMCC, rinsed with ddH2O and spin-dried at 1,500 rpm for 10 min.

A library of 10,410 random 20-mer sequences was spotted in duplicates onto the SMCC-activated surfaces with a robotic printer (Telechem Nanoprint 60 using 48 Telechem series SMP2 style titanium pins). Briefly, the peptide library was synthesized by Alta Biosciences Ltd (Birmingham, UK) based on randomized amino acid sequences provided by in-house software. The conserved C-terminal-GSC sequence of each peptide was used as a linker to mediate coupling to the array surface. The sequence of the other 17 amino acids was generated by a random peptide sequence generator using 19 amino acids, cysteine excluded, resulting in 95% functional diversity.

After printing was complete, the slides were incubated for 12 hours at 60% humidity, incubated for 30 min in 95% TFA to remove excess of deposited peptides, rinsed five times with ddH2O, and spin-dried at 1,500 rpm for 10 min.

Microarray screening. Aliquots of the bacterial cultures were centrifuged to isolate the cells and were washed twice with an equal volume of PBS buffer (pH 7.2). After each wash, bacteria were resuspended and centrifuged. Cells were fluorescently stained with SYTO 9 green-fluorescent cell-permeable nucleic acid dye (Molecular Probes, Eugene, Oreg.). Dye was added to a 500 microliter suspension of bacteria at a concentration of 20 μM in PBS buffer and shaken for 1 hr at 37 degrees Centigrade. After incubation, the cell suspension was centrifuged, isolated, and washed twice with 1 ml PBS buffer. After staining, samples were directly applied to the arrays (dye that was not cell associated does not fluoresce). Each array was equipped with an Agilent hybridization chamber, and then 450 microliter solution of E. coli in PBS buffer supplemented with 0.03% FBS was added. The arrays were gently shaken on a platform for 1 hr at 37 degrees Centigrade temperature. After incubation, the hybridization chamber was removed, and unbound cells were washed away from the array by dipping it into a 50 ml solution of the hybridization buffer. This step was repeated twice, and the buffer was changed between washings. A final wash with nanopure water was used to remove salts from the array. Slides were then briefly centrifuged at 1500 rpm for 2 min to dry the arrays.

Data analysis. The arrays were scanned using a laser scanner (PERKIN-ELMER PROSCANARRAY HT) and quantified using GENEPIX PRO6.0 (Molecular Devices, Inc.) in order to determine the fluorescence intensity of each probe and identify its corresponding peptide sequence. A statistical comparison of microarray data was done with GENESPRING GX 7.3 (AGILENT, INC, Palo Alto, Calif., USA) by importing the image-processed data from GENEPIX PRO6.0. Median signal intensities were used in the calculations. For statistical comparisons, each slide was normalized to 50th percentile. Also, measurements of less than 0.01 were set to 0.01. Results were represented on a heatmap in order to be able to compare the difference in intensities with and without excess LPS.

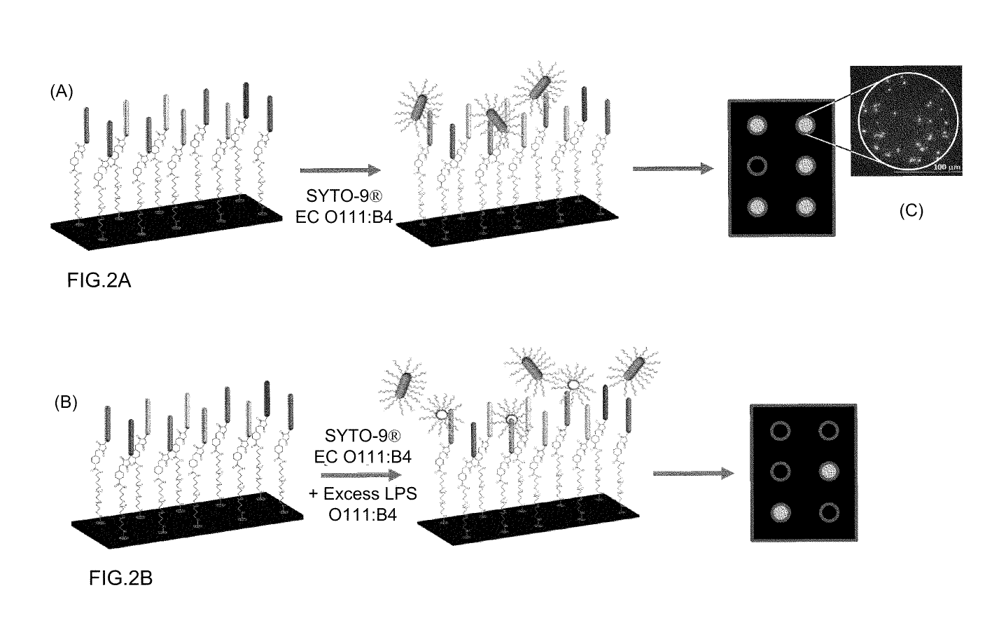

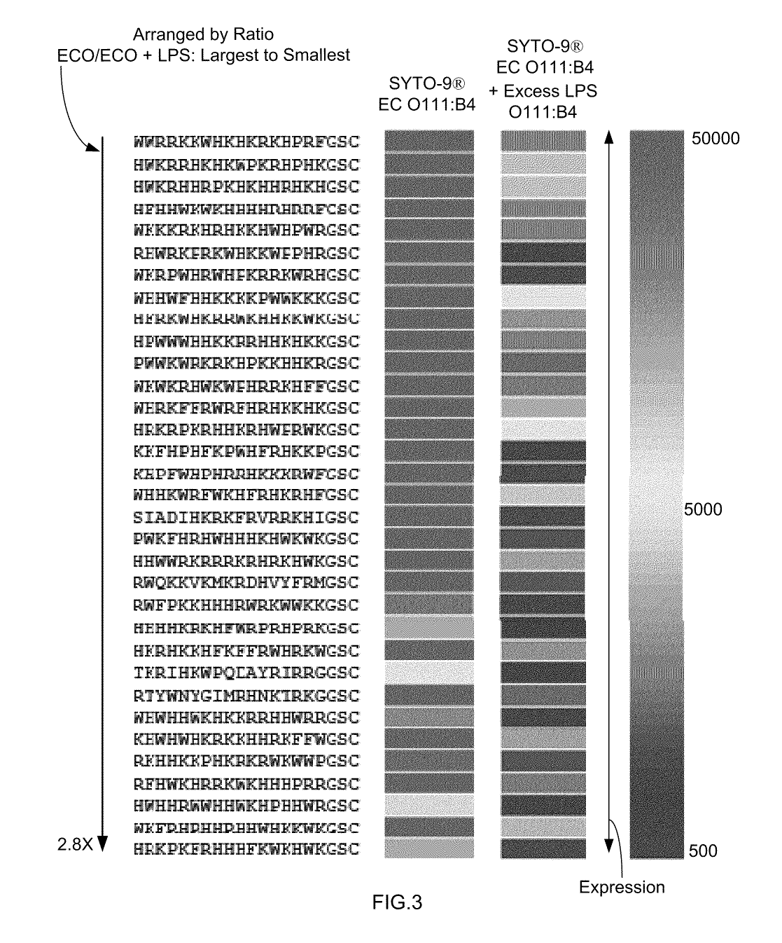

LPS competition assay. Since approximately 90% of bacterial surface is comprised of LPS it was expected that bacteria will bind to random peptide microarray mostly through LPS. In order to demonstrate that peptides on the microarray indeed bind bacteria through LPS competition assays were conducted. Same number of cells stained with SYTO-9® was used to probe the microarrays with and without excess non-labeled LPS from the same strain (FIG. 2). 54 peptides were found that showed >2× decrease in signal in presence of LPS. The heat map in FIG. 3 shows that intensities of signals for peptides that bound E. coli with and without excess LPS are 2.8-8.7-fold different from each other, indicating effective inhibition of bacterial binding by free LPS.

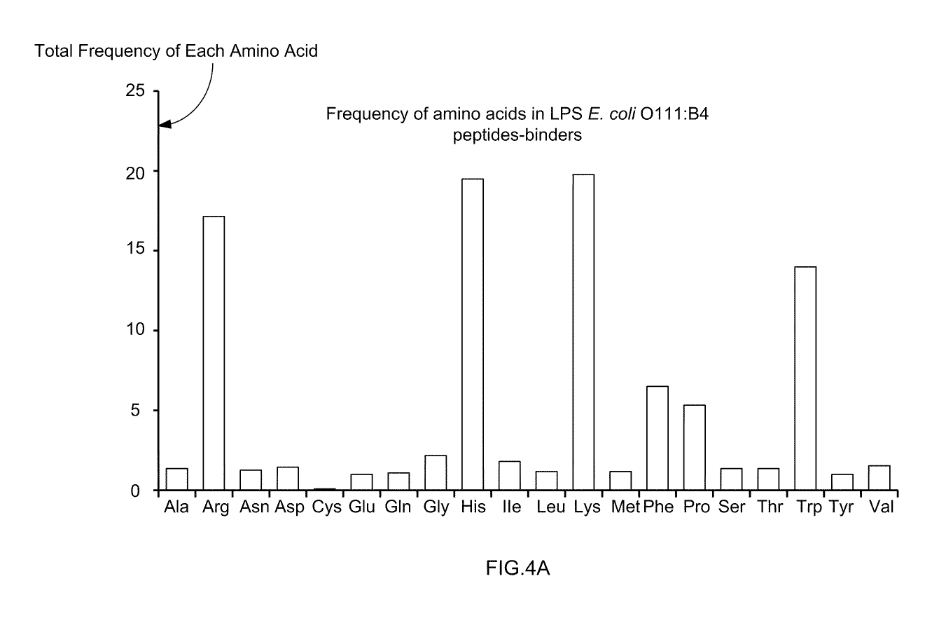

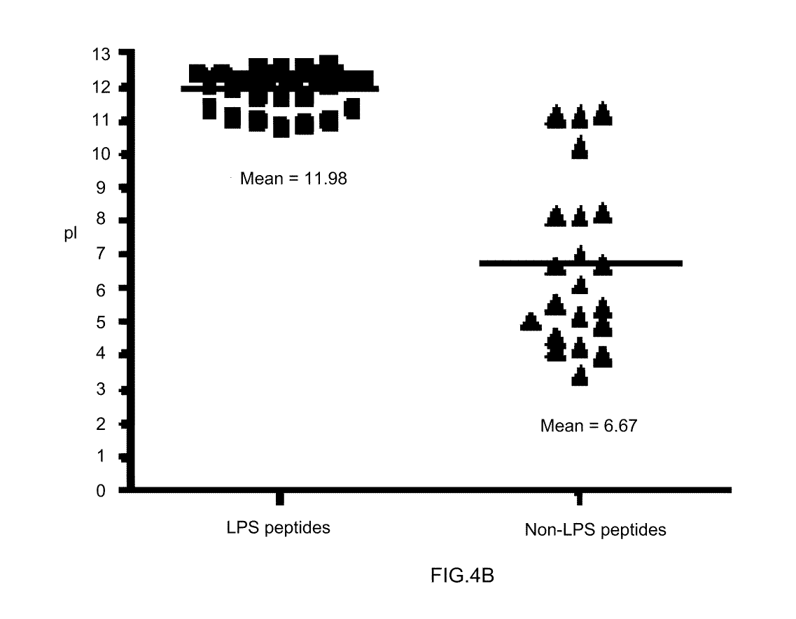

The physical properties of LPS binders are presented in Table 2. All selected peptides appeared to be cationic amphipathic peptides with the prevalence of phenylalanine, histidine, lysine, arginine, and tryptophan; amino acids commonly present in natural antimicrobial peptides. Frequencies of amino acids in the sequences of those peptides ranged as shown on FIG. 4A and pl distribution is shown in FIG. 4B. Despite prevalence of LPS on the membrane of gram-negative bacteria there are some proteins which possibly might be a target for peptides on the array. Some of the signals were not suppressed with LPS and could be an evidence of either electrostatic dye binding or other peptide-surface component interactions. Properties of peptides with stable signals in competition assay were different from those presented in Table 2. For instance, charge on average tends to be mostly neutral with only a few exceptions.

TABLE 2

| SEQ | | | | | | | Ratio |
|---|---|---|---|---|---|---|---|
| ID | Peptide | | | Negative | | Aliphatic | ECO/ |
| NO: | sequence | MW | pI | residues | Positive | index | (ECO + LPS) |
| 1 | WWRRKKWHKHK | 2745.2 | 12.02 | 0 | 9 | 0 | 18.75948 |
| | RKHPRFGSC | | | | | | |
| 2 | HWKRRHKHKWP | 2627 | 11.76 | 0 | 8 | 0 | 13.22173 |
| | KRHPHKGSC | | | | | | |
| 3 | HWKRHHRPKHK | 2627 | 11.75 | 0 | 7 | 0 | 11.47856 |
| | HHRHKHGSC | | | | | | |
| 4 | HFHHWKWKHHH | 2754.1 | 11.72 | 0 | 5 | 0 | 11.28025 |
| | HRHRRFGSC | | | | | | |
| 5 | WKKKRKHRHKK | 2707.2 | 11.76 | 0 | 9 | 0 | 10.30474 |
| | HWHPWRGSC | | | | | | |
| 6 | RHWRKPRKWHK | 2664.1 | 12.02 | 0 | 8 | 0 | 9.880503 |
| | KWPPHRGSC | | | | | | |
| 7 | WKRPWHRWHPK | 2781.2 | 12.18 | 0 | 8 | 0 | 9.610698 |
| | RRKWRHGSC | | | | | | |
| 8 | WHHWFHHKKKK | 2700.2 | 10.41 | 0 | 7 | 0 | 8.832187 |
| | PWWKKKGSC | | | | | | |
| 9 | HFRKWHKRRWK | 2757.2 | 11.76 | 0 | 9 | 0 | 8.434816 |
| | HHKKWKGSC | | | | | | |
| 10 | HPWWWHHKKRR | 2697.1 | 11.22 | 0 | 7 | 0 | 5.344861 |
| | HHKHKKGSC | | | | | | |
| 11 | PWWKWRKRKHP | 2667.1 | 11.76 | 0 | 9 | 0 | 4.687001 |
| | KKHHKRGSC | | | | | | |
| 12 | WKWKRHWKWP | 2794.2 | 11.75 | 0 | 7 | 0 | 4.576375 |
| | HRRKHFFGSC | | | | | | |
| 13 | WHRKFFRWRFH | 2765.2 | 12.02 | 0 | 8 | 0 | 4.547302 |
| | RHKKHKGSC | | | | | | |
| 14 | HRKRPKRHHKRH | 2674.1 | 12.19 | 0 | 9 | 0 | 4.429209 |
| | WPRWKGSC | | | | | | |
| 15 | KKFHPHFKPWHF | 2530 | 10.6 | 0 | 6 | 0 | 4.303266 |
| | RHKKPGSC | | | | | | |
| 16 | KHPFWHPHRRH | 2656.1 | 11.75 | 0 | 7 | 0 | 4.283159 |
| | KKKRWFGSC | | | | | | |
| 17 | WHHKWRFWKHF | 2804.2 | 11.74 | 0 | 6 | 0 | 4.216433 |
| | RHKRHFGSC | | | | | | |
| 18 | SIADIHKRKFRVR | 2407.8 | 11.57 | 1 | 7 | 78 | 4.040411 |
| | RKHIGSC | | | | | | |
| 19 | PWKFHRHWHHH | 2746.1 | 10.48 | 0 | 5 | 0 | 3.643969 |
| | KHWKWKGSC | | | | | | |
| 20 | HHWWRKRRRKR | 2822.3 | 12.31 | 0 | 10 | 0 | 3.527244 |
| | HRKHWKGSC | | | | | | |
| 21 | RWQKKVKMKRD | 2584.1 | 10.56 | 1 | 7 | 29 | 3.525649 |
| | HVYFRMGSC | | | | | | |
| 22 | RWFPKKHHHRW | 2775.2 | 11.76 | 0 | 8 | 0 | 3.506421 |
| | RKWWKKGSC | | | | | | |
| 23 | HHHHKRKHFWR | 2625 | 12.01 | 0 | 7 | 0 | 3.493433 |
| | PRHPRKGSC | | | | | | |
| 24 | HKRHKKHFKFFR | 2737.2 | 11.76 | 0 | 8 | 0 | 3.427726 |
| | WHRKWGSC | | | | | | |
| 25 | TKRIHKWPQDAY | 2428.8 | 10.94 | 1 | 6 | 44 | 3.354743 |
| | RIRRGGSC | | | | | | |
| 26 | RTYWNYGIMRHN | 2428.7 | 10.45 | 0 | 5 | 19.5 | 3.331281 |
| | KTRKGGSC | | | | | | |
| 27 | WHWHHWKHKKR | 2842.2 | 12.01 | 0 | 7 | 0 | 3.260905 |
| | RHHWRRGSC | | | | | | |
| 28 | KHWHWHKRKKH | 2757.2 | 11.22 | 0 | 7 | 0 | 3.0313 |
| | HRKFFWGSC | | | | | | |
| 29 | RKHHKKPHKRKR | 2667.1 | 11.76 | 0 | 9 | 0 | 3.020833 |
| | WKWWPGSC | | | | | | |
| 30 | RFHWKHRRKWK | 2733.1 | 12.18 | 0 | 8 | 0 | 2.943605 |
| | HHHPRRGSC | | | | | | |
| 31 | HWHHRWWHHW | 2831.1 | 10.86 | 0 | 3 | 0 | 2.853832 |
| | KHPHHWRGSC | | | | | | |
| 32 | WKFRHRHHRHH | 2775.2 | 11.75 | 0 | 7 | 0 | 2.844429 |
| | WHKKWKGSC | | | | | | |
| 33 | HRKPKFRHHHFK | 2668.1 | 11.22 | 0 | 7 | 0 | 2.786033 |
| | WKHWKGSC (QF8) | | | | | | |
| 34 | RRWWRKRFWHH | 2918.4 | 12.4 | 0 | 9 | 0 | 2.423464 |
| | RWHRRKGSC | | | | | | |
| 35 | HRWWFKKKHRF | 2902.4 | 12.02 | 0 | 8 | 0 | 2.403501 |
| | RWWKRWGSC | | | | | | |
| 36 | RFFFHKWRRWR | 2760.2 | 12.4 | 0 | 9 | 0 | 2.377551 |
| | RRKPPRGSC | | | | | | |
| 37 | RWRRHKHFKRP | 2694.1 | 12.31 | 0 | 10 | 0 | 2.374598 |
| | HRKHKRGSC | | | | | | |
| 38 | WWHHKWFKHKK | 2797.2 | 10.6 | 0 | 6 | 0 | 2.348657 |
| | FWRHKFGSC | | | | | | |
| 39 | WKPWFHPRKHH | 2734.1 | 11.11 | 0 | 5 | 0 | 2.334201 |
| | KWRHWHGSC | | | | | | |
| 40 | KHKWWRWHKW | 2903.3 | 11.17 | 0 | 6 | 0 | 2.322466 |
| | HWWFRKHGSC | | | | | | |
| 41 | KWKHHKKFRKRK | 2718.2 | 11.76 | 0 | 9 | 0 | 2.315513 |
| | RHWFHGSC | | | | | | |
| 42 | HWHPRWHKHKF | 2774.1 | 11.11 | 0 | 5 | 0 | 2.273486 |
| | KHWRWHGSC | | | | | | |
| 43 | WRFKHFRWKRF | 2737.2 | 11.76 | 0 | 8 | 0 | 2.262656 |
| | KHHHKKGSC | | | | | | |
| 44 | IYNKRSQVRRKG | 2446.9 | 10.72 | 0 | 8 | 34 | 2.168758 |
| | FKKKYGSC | | | | | | |
| 45 | RKRKWHHPWHW | 2840.3 | 12.31 | 0 | 9 | 0 | 2.135974 |
| | RRKRRWGSC | | | | | | |
| 46 | RHWRPKFRKFR | 2850.3 | 12.18 | 0 | 7 | 0 | 2.124858 |
| | WWRWHHGSC | | | | | | |
| 47 | FHKKRHFKRRHK | 2814.3 | 12.02 | 0 | 8 | 0 | 2.113703 |
| | FWWWRGSC | | | | | | |
| 48 | HKWHRKKFHKH | 2650.1 | 11.27 | 0 | 8 | 0 | 2.112385 |
| | RFKHHKGSC | | | | | | |
| 49 | WFWKHKKWRRH | 2833.3 | 11.75 | 0 | 7 | 0 | 2.104218 |
| | PRKWHWGSC | | | | | | |
| 50 | EPKLWFKPRRGG | 2467.8 | 10.94 | 1 | 6 | 19.5 | 2.035168 |
| | YRHRHGSC | | | | | | |
| 51 | HRKWHWPWKW | 2832.3 | 11.75 | 0 | 7 | 0 | 2.033291 |
| | PWKRKRWGSC | | | | | | |
| 52 | RPKPHRHWKPKF | 2555.9 | 11.11 | 0 | 5 | 0 | 2.031414 |
| | PHHHWGSC | | | | | | |
| 53 | WRRSTPVGPWT | 2368.7 | 10.86 | 0 | 3 | 34 | 1.973999 |
| | WFGKFLGSC | | | | | | |
| 54 | RRFRHWWRWRK | 2956.4 | 12.55 | 0 | 11 | 0 | 1.91419 |
| | RRRKWRGSC | | | | | | |

Multivalent constructs. It had been previously shown that most of LPS-binding peptides were also effective growth inhibitors of corresponding bacteria. Here it was intended to test if their effectiveness can be further enhanced by the multivalent presentation of the peptides. To this end, peptide QF8 (HRKPKFRHHHFKWKHWKGSC SEQ ID NO: 33, Table 2) was resynthesized and purified (>95%) by HPLC. Several multimeric Constructs were prepared (FIG. 4) containing peptide QF8 and, as a negative control, peptide NEG-1 (EFSNPTAQVFPDFWMSDGSC) that was shown to bind neither to the bacteria nor to LPS (Morales et al., 2009). Binding efficiency of the selected peptides could be enhanced by multivalent presentation of the peptides.

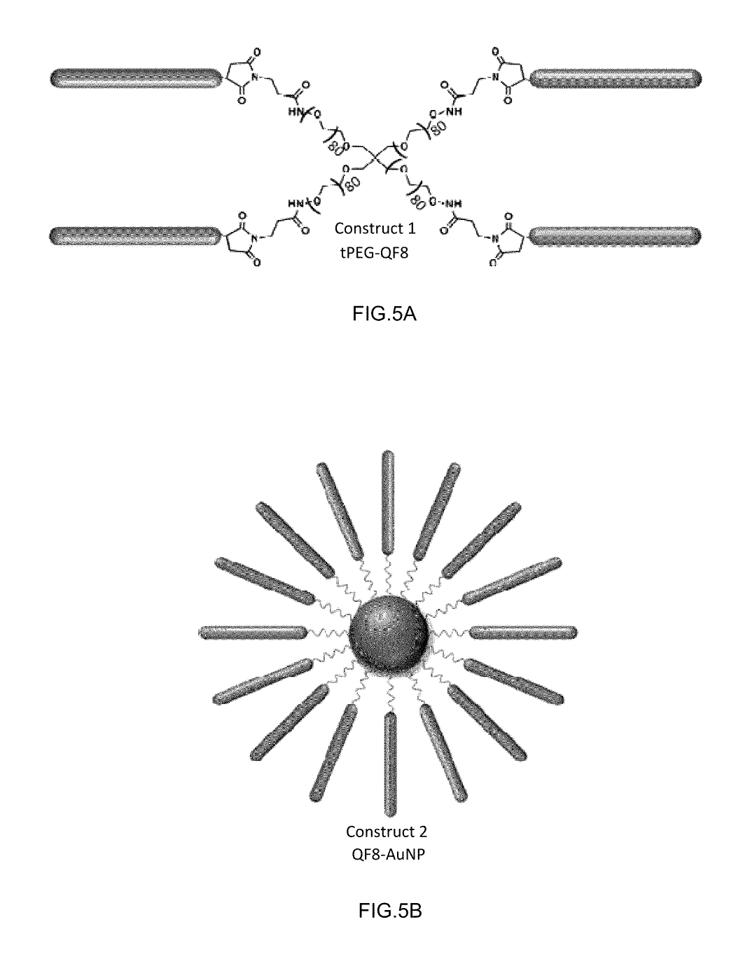

Synthesis of tetravalent Construct 1 (FIG. 5). To 1 mg (0.05 μmols) of tetravalent mPEG-maleimide (NOF America Corporation, MW 20 kDa) in 500 μL of 1×PBS, 4.2 mg (1.5 μmols, 7.5-8-fold excess over each maleimide) of a peptide were added. After reacting overnight at room temperature, the mixture was purified by dialyzing against ddH2O using a 10K dialyzing cassette and lyophilized. The functionalization was confirmed by MALDI-TOF.

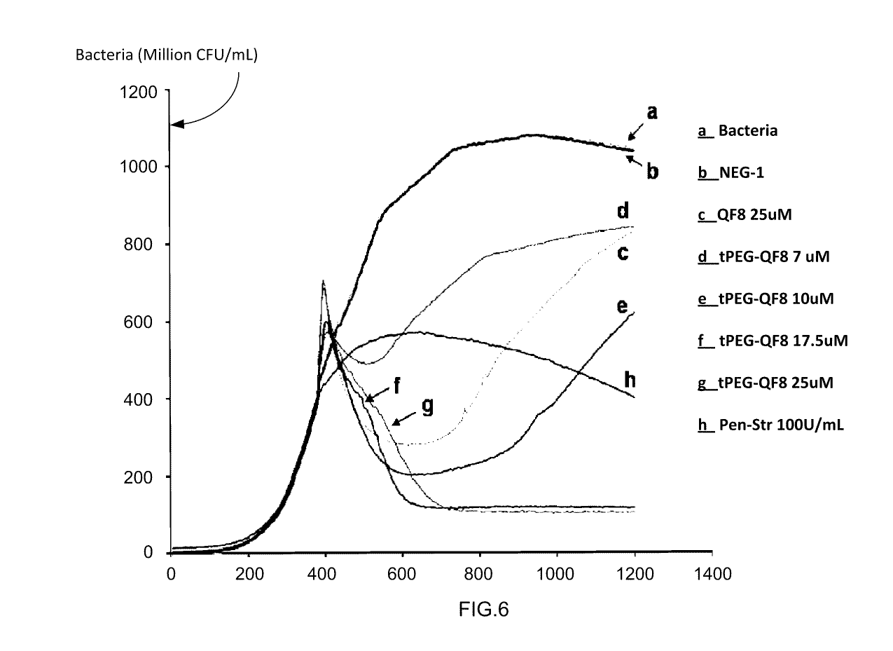

In vitro growth inhibition assays. Growth of E. coli O111:B4 was assessed in the presence of different concentrations of NEG-1, QF8, and Construct 1 using turbidity at 600 nm. Peptide treatments were added during the mid-logarithmic phase at the following concentrations: QF8 peptide at 25 micrometers, NEG-1 peptide at 100 micrometers, Construct 1 at 7, 10, and 25 micrometers, and, as a positive control, penicillin-streptomycin at recommended concentration of 100 units/mL. The growth curves show no changes between the untreated bacteria and the negative control but show a dramatic decrease in bacterial count with increasing concentrations of the monomeric QF8 peptide and especially its tetrameric Construct 1 (FIG. 6).

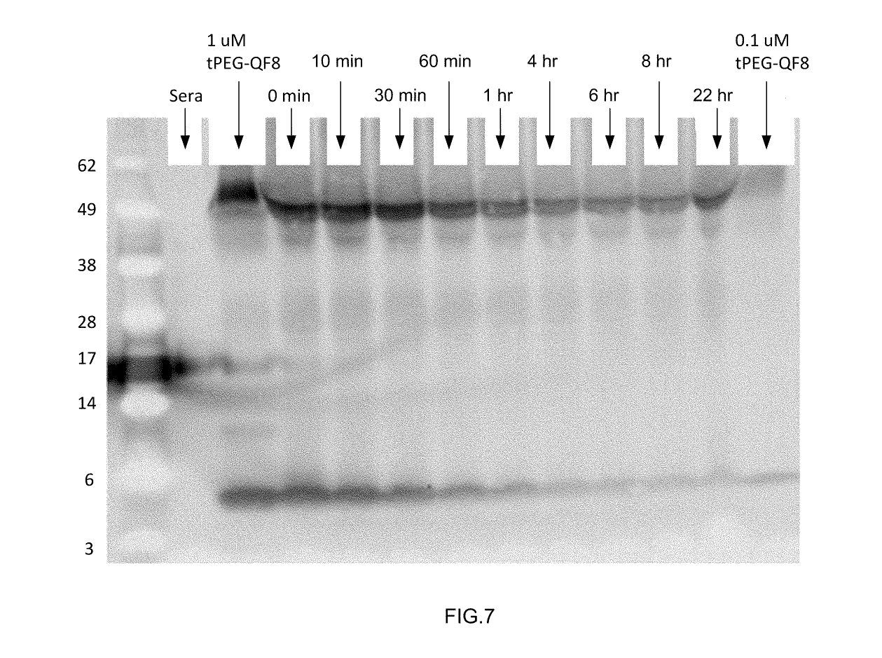

Serum stability of Construct 1. Over the last decade, there was a considerable interest in developing antimicrobial peptides as intravenously administered antibiotics. In this respect, a common criticism of peptide-based drugs has been their susceptibility to endogenous proteases. Thus, after successful demonstration of Construct 1's antibacterial activity, the next critical test for therapeutic potential was to measure the serum stability of the construct. To test the in vitro stability of Construct 1 we used a serum-incubation and western blot analysis. Construct 1 was incubated in fresh human serum at 37° C. and pH=7.4 with a starting concentration of 10 μM. Samples were withdrawn at predetermined time points and each sample diluted 20-fold with loading buffer prior to analysis by western blot (FIG. 7). We found that Construct 1 resisted proteolysis for more than 8 hours. These data demonstrate that the tetravalent constructs are promising antimicrobial candidates.

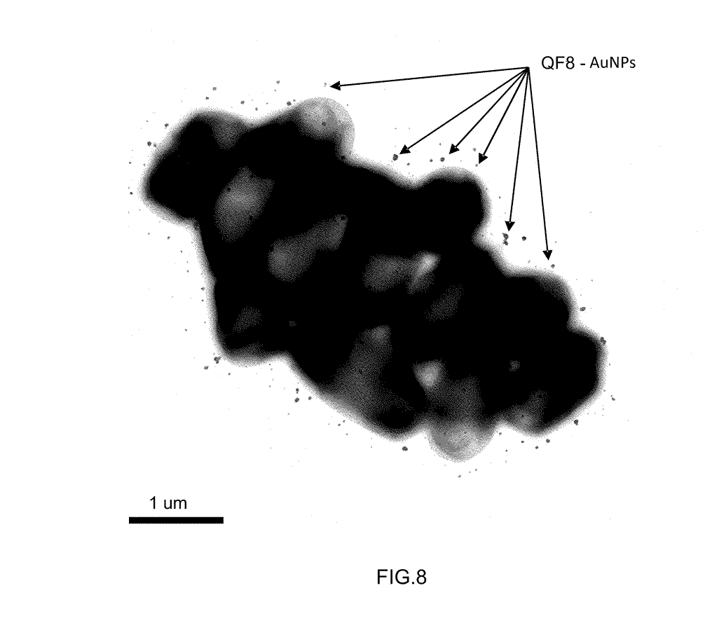

Bacterial agglutination. Agglutination of E. coli was observed in the presence of QF8-AuNPs but not with the control NEG1-AuNPs and was not seen in both cases in the absence of bacteria (FIG. 8). Since antimicrobial peptides typically consist of positively charged amino acid residues and the bacteria typically carry negative charge, the inventor tested if the positive charge was responsible for the agglutination. To test this, cysteamine-modified gold nanoparticles (Cys-AuNP) were synthesized that had charge similar to QF8-AuNPs, +32 mV vs +36 mV respectively. Adding similar concentration of Cys-AuNPs (absorbance at 550 nm) to the same number of bacteria did not induce agglutination but resulted in a red shift of plasmon resonance indicative of the nanoparticles assembling at the surface of bacteria, but not agglutinating it. The similar effect was recently reported for gold nanoparticles aggregating at the surface of a virus (Niikura et al., 2009). In contrast to QF8-AuNP and NEG1-AuNP the Cys-AuNPs entirely disappeared from the bacterial solution in a few hours; it is possible that the cysteamine was consumed by the bacteria resulting in precipitation of unprotected nanoparticles.

To test if hydrophobic component played a role in QF8-AuNP induced agglutination, the reaction was performed in the presence of 0.25% Tween-20. Preliminary experiments showed that this concentration of the non-ionic detergent did not affect the growth of E. coli. QF8-AuNPs induced strong agglutination. This agglutination was significantly reduced by the detergent. These results suggest that hydrophobic interactions play a role in QF8-AuNP induced agglutination.

Magnetic Separation. Magnetic microbeads have been widely used as a powerful separation tool for such targets as cells, bacteria, and biochemical assays. The ability to physically separate cells from heterogeneous mixtures can be used for enrichment and purification of target cells. Multiple researchers have used lectin-modified magnetic beads for separation of bacteria, for separation of leukemia cells based on leukemia-specific carbohydrate antigens, as purging agents in myeloma, for large-scale extraction and purification of human pancreatic islets, etc.

Compared to flow cytometric cell-sorting, magnetic-based separations offer the clear advantage of rapid, bulk processing of large number of cells. Here, specific glycan binding properties of lectins or anticarbohydrate antibodies form a basis for their use as cell capture and separation tools. One clear disadvantage of using antibodies and lectins for cell separation is their cost and bulkiness. Smaller, cheaper molecules would be a welcome addition to the armamentarium of cell separation tools. Short peptides are ideal in this respect as they are easy to synthesize, manipulate, and conjugate to a variety of scaffolds. Multiple copies of QF8 peptide were cross-linked to the surface of amino-functionalized magnetic DYNABEADS® by using a hetero-bifunctional amino/thiol reactive linker. The linker was first conjugated to the surface amino groups on DYNABEADS® to yield a maleimide-modified surface which was then reacted with cysteine thiols of the peptides. The peptide-functionalized magnetic beads were incubated with a mixture of 0111:84 E. coli cells. The inspection of the confocal microscopy images clearly indicate that the bacteria bound to the magnetic beads. Cells bound by the conjugates were magnetically separated and thoroughly washed to remove any unbound cells and were subjected to TEM imaging that showed agglutination aggregates similar to the ones shown in FIG. 8.

Preparation of QF8-functionalized gold nanoparticles. Nanoparticles were prepared according to known procedures with slight modifications. A solution of 2 mg of QF8 peptide in 400 microliters in ultrapure water, or 400 microliters of 213 mM (16.4 mg/ml) of cysteamine in ultrapure water was added to 40 mL of a solution of 1.42 mM HAuCl4 (0.625 mg/mL) in ultrapure water. The mixture turned yellow and translucent upon addition of QF8/cysteamine and was stirred vigorously for 10 min. A freshly prepared solution (30 microliters) of 10 mM NaBH4 was quickly injected into the reaction mixture with a micropipette. The solution immediately turned light brown. After vigorous stirring for 15-30 min, the solution became wine red. The solution of nanoparticles can be stored in the dark at 4 degrees Centigrade for up to one week; it tends to become darker over longer periods of time indicating transient nature of the formed nanoparticles.

Preparation of QF8-functionalized magnetic nanoparticles. To 500 microliters of amino functionalized iron oxide nanocrystals (Ocean NanoTech, Inc) solved in 200 microliters of 1×PBS buffer, sulfo-SMCC (2 mg) was added and the reaction was shaken at 1400 rpm for 30 min at RT. The mixture was centrifuged at 14000 rpm for 15 min and the supernatant discarded. The particles were resuspended in 200 microliters of 1×PBS with sonication, peptide QF8 (2 mg) was immediately added and the reaction was shaken at 1400 rpm overnight at RT. To remove non reacted peptide, the mixture was centrifuged at 14000 for 20 min, and after removing the supernatant, the particles were washed 2× with ddH2O. Finally, the nanocrystals were suspended in 500 microliters of water.

Preparation of QF8-functionalized magnetic beads. 335 microliters of DYNABEADS® M-280 Tosyl-activated (Invitrogen, Inc.) solution was centrifuged and the beads precipitated. After removing the water, 2 mg of QF8 peptide solved in 335 microliters of 1×PBS buffer were added. The reaction was shaken at 1400 rpm at RT for 16 hours, and then the particles washed 3× with ddH2O. The resultant peptide functionalized magnetic beads were resuspended on 500 microliters of ddH2O.

Bacterial agglutination. Bacterial agglutination was performed using live E. coli 0111:84 cells grown to mid log phase, then centrifuged and resuspended in saline to 5×10⁸ bacteria/ml. QF8-AuNPs were added to the cell preparations. The agglutination of the cells occurred instantly and was characterized by a coarse granular bacterial clumping.

In vitro inhibition assays. E. coli 0111:84 (ATCC 33780) were grown aerobically at 30 degrees Centigrade in ATCC nutrient broth 3. Growth was monitored as turbidity at 600 nm. Experiments were performed at 30 degrees Centigrade in microtiter plates with 100 microliters of culture using a Spectra Max Plus microtiter plate reader with Soft Max Pro Software (Molecular devices). The turbidity was measured every 5 minutes and the plate was shaken for 30 seconds before each measurement. Bacteria concentration was calculated according to the McFarland general method using the absorbance at 600 nm of a series of mixtures of 1% barium chloride and 1% sulfuric acid.

What is claimed is:

1. An isolated glycan binding peptide complex comprising two or more glycan binding peptides operatively coupled to each other.

2. The glycan binding peptide complex of claim 1, wherein the complex comprises at least 4 glycan binding peptides.

3. The glycan binding peptide complex of claim 1, wherein the glycan binding peptides are coupled via a linker moiety.

4. The glycan binding peptide complex of claim 1, wherein the linker moiety is a polymer.

5. The glycan binding peptide complex of claim 4, wherein the linker moiety is polyethylene glycol (PEG), polyvinyl alcohol (PVA), polyacrylamide (PLA), polylactic acid (PL), polyglycolide (PG), poly(lactic-co-glycolic) acid (PLGA), poly(glycine), poly(proline), amino acid polymer and/or nucleic acid polymer.

6. The glycan binding peptide complex of claim 4, wherein the polymer is a polyethylene glycol polymer or polyethyleneimine polymer.

7. The glycan binding peptide complex of claim 4, wherein the linker is a multivalent polymer that can be coupled to at least 3 glycan binding peptides.

8. The glycan binding peptide complex of claim 7, wherein the glycan binding peptides comprise the same amino acid sequence.

9. The glycan binding peptide complex of claim 1, wherein the linker is coupled to a particle.

10. The glycan binding peptide complex of claim 1, wherein the particle is a magnetic particle.

11. A peptide microarray comprising a plurality of glycan binding peptides each operatively coupled to a substrate by a linker moiety that is at least 2 to 1,000 repeating units in length.

12. The peptide microarray of claim 11, wherein the linker moiety is a polymer.

13. The peptide microarray of claim 12, wherein the linker moiety is polyethylene glycol (PEG), polyvinyl alcohol (PVA), polyacrylamide (PLA), polylactic acid (PL), polyglycolide (PG), poly(lactic-co-glycolic) acid (PLGA), poly(glycine), poly(proline), amino acid polymer and/or nucleic acid polymer.

14. The peptide microarray of claim 12, wherein the polymer is a polyethyleneimine polymer.

15. The peptide microarray of claim 11, wherein the substrate is a bead.

16. The peptide microarray of claim 11, wherein the substrate is a planar surface.

17. A method of attenuating bacterial growth comprising contacting a bacteria with a glycan binding peptide complex of claim 1.

18. The method of claim 17, wherein the bacteria is on a surface or in a solution.

19. The method of claim 17, wherein the bacteria are in a subject.

20. A method of evaluating a substance for the presence of a microbe comprising contacting the substance with a peptide microarray or a peptide complex comprising a plurality glycan binding peptide operatively coupled to a substrate or multivalent linker, wherein the glycan binding peptide is coupled to an array by a linker that is at least 0.5 micrometers in length.


SEQUENCE LISTING (55 sequences)

SEQ ID NO: 1 - protein, 20 aa, Artificial Sequence
  SYNTHETIC PEPTIDE
WWRRKKWHKHKRKHPRFGSC

SEQ ID NO: 2 - protein, 20 aa, Artificial Sequence
  SYNTHETIC PEPTIDE
HWKRRHKHKWPKRHPHKGSC

SEQ ID NO: 3 - protein, 20 aa, Artificial Sequence
  SYNTHETIC PEPTIDE
HWKRHHRPKHKHHRHKHGSC

SEQ ID NO: 4 - protein, 20 aa, Artificial Sequence
  SYNTHETIC PEPTIDE
HFHHWKWKHHHHRHRRFGSC

SEQ ID NO: 5 - protein, 20 aa, Artificial Sequence
  SYNTHETIC PEPTIDE
WKKKRKHRHKKHWHPWRGSC

SEQ ID NO: 6 - protein, 20 aa, Artificial Sequence
  SYNTHETIC PEPTIDE
RHWRKPRKWHKKWPPHRGSC

SEQ ID NO: 7 - protein, 20 aa, Artificial Sequence
  SYNTHETIC PEPTIDE
WKRPWHRWHPKRRKWRHGSC

SEQ ID NO: 8 - protein, 20 aa, Artificial Sequence
  SYNTHETIC SEQUENCE
WHHWFHHKKKKPWWKKKGSC

SEQ ID NO: 9 - protein, 20 aa, Artificial Sequence
  SYNTHETIC PEPTIDE
HFRKWHKRRWKHHKKWKGSC

SEQ ID NO: 10 - protein, 20 aa, Artificial Sequence
  SYNTHETIC PEPTIDE
HPWWWHHKKRRHHKHKKGSC

SEQ ID NO: 11 - protein, 20 aa, Artificial Sequence
  SYNTHETIC PEPTIDE
PWWKWRKRKHPKKHHKRGSC

SEQ ID NO: 12 - protein, 20 aa, Artificial Sequence
  SYNTHETIC PEPTIDE
WKWKRHWKWPHRRKHFFGSC

SEQ ID NO: 13 - protein, 20 aa, Artificial Sequence
  SYNTHETIC PEPTIDE
WHRKFFRWRFHRHKKHKGSC

SEQ ID NO: 14 - protein, 20 aa, Artificial Sequence
  SYNTHETIC PEPTIDE
HRKRPKRHHKRHWPRWKGSC

SEQ ID NO: 15 - protein, 20 aa, Artificial Sequence
  SYNTHETIC PEPTIDE
KKFHPHFKPWHFRHKKPGSC

SEQ ID NO: 16 - protein, 20 aa, Artificial Sequence
  SYNTHETIC PEPTIDE
KHPFWHPHRRHKKKRWFGSC

SEQ ID NO: 17 - protein, 20 aa, Artificial Sequence
  SYNTHETIC PEPTIDE
WHHKWRFWKHFRHKRHFGSC

SEQ ID NO: 18 - protein, 20 aa, Artificial Sequence
  SYNTHETIC PEPTIDE
SIADIHKRKFRVRRKHIGSC

SEQ ID NO: 19 - protein, 20 aa, Artificial Sequence
  SYNTHETIC PEPTIDE
PWKFHRHWHHHKHWKWKGSC

SEQ ID NO: 20 - protein, 20 aa, Artificial Sequence
  SYNTHETIC PEPTIDE
HHWWRKRRRKRHRKHWKGSC

SEQ ID NO: 21 - protein, 20 aa, Artificial Sequence
  SYNTHETIC PEPTIDE
RWQKKVKMKRDHVYFRMGSC

SEQ ID NO: 22 - protein, 20 aa, Artificial Sequence
  SYNTHETIC PEPTIDE
RWFPKKHHHRWRKWWKKGSC

SEQ ID NO: 23 - protein, 20 aa, Artificial Sequence
  SYNTHETIC PEPTIDE
HHHHKRKHFWRPRHPRKGSC

SEQ ID NO: 24 - protein, 20 aa, Artificial Sequence
  SYNTHETIC PEPTIDE
HKRHKKHFKFFRWHRKWGSC

SEQ ID NO: 25 - protein, 20 aa, Artificial Sequence
  SYNTHETIC PEPTIDE
TKRIHKWPQDAYRIRRGGSC

SEQ ID NO: 26 - protein, 20 aa, Artificial Sequence
  SYNTHETIC PEPTIDE
RTYWNYGIMRHNKTRKGGSC

SEQ ID NO: 27 - protein, 20 aa, Artificial Sequence
  SYNTHETIC PEPTIDE
WHWHHWKHKKRRHHWRRGSC

SEQ ID NO: 28 - protein, 20 aa, Artificial Sequence
  SYNTHETIC PEPTIDE
KHWHWHKRKKHHRKFFWGSC

SEQ ID NO: 29 - protein, 20 aa, Artificial Sequence
  SYNTHETIC PEPTIDE
RKHHKKPHKRKRWKWWPGSC

SEQ ID NO: 30 - protein, 20 aa, Artificial Sequence
  SYNTHETIC PEPTIDE
RFHWKHRRKWKHHHPRRGSC

SEQ ID NO: 31 - protein, 20 aa, Artificial Sequence
  SYNTHETIC PEPTIDE
HWHHRWWHHWKHPHHWRGSC

SEQ ID NO: 32 - protein, 20 aa, Artificial Sequence
  SYNTHETIC PEPTIDE
WKFRHRHHRHHWHKKWKGSC

SEQ ID NO: 33 - protein, 20 aa, Artificial Sequence
  SYNTHETIC PEPTIDE
HRKPKFRHHHFKWKHWKGSC

SEQ ID NO: 34 - protein, 20 aa, Artificial Sequence
  SYNTHETIC PEPTIDE
RRWWRKRFWHHRWHRRKGSC

SEQ ID NO: 35 - protein, 20 aa, Artificial Sequence
  SYNTHETIC PEPTIDE
HRWWFKKKHRFRWWKRWGSC

SEQ ID NO: 36 - protein, 20 aa, Artificial Sequence
  SYNTHETIC PEPTIDE
RFFFHKWRRWRRRKPPRGSC

SEQ ID NO: 37 - protein, 20 aa, Artificial Sequence
  SYNTHETIC PEPTIDE
RWRRHKHFKRPHRKHKRGSC

SEQ ID NO: 38 - protein, 20 aa, Artificial Sequence
  SYNTHETIC PEPTIDE
WWHHKWFKHKKFWRHKFGSC

SEQ ID NO: 39 - protein, 20 aa, Artificial Sequence
  SYNTHETIC PEPTIDE
WKPWFHPRKHHKWRHWHGSC

SEQ ID NO: 40 - protein, 20 aa, Artificial Sequence
  SYNTHETIC PEPTIDE
KHKWWRWHKWHWWFRKHGSC

SEQ ID NO: 41 - protein, 20 aa, Artificial Sequence
  SYNTHETIC PEPTIDE
KWKHHKKFRKRKRHWFHGSC

SEQ ID NO: 42 - protein, 20 aa, Artificial Sequence
  SYNTHETIC PEPTIDE
HWHPRWHKHKFKHWRWHGSC

SEQ ID NO: 43 - protein, 20 aa, Artificial Sequence
  SYNTHETIC PEPTIDE
WRFKHFRWKRFKHHHKKGSC

SEQ ID NO: 44 - protein, 20 aa, Artificial Sequence
  SYNTHETIC PEPTIDE
IYNKRSQVRRKGFKKKYGSC

SEQ ID NO: 45 - protein, 20 aa, Artificial Sequence
  SYNTHETIC PEPTIDE
RKRKWHHPWHWRRKRRWGSC

SEQ ID NO: 46 - protein, 20 aa, Artificial Sequence
  SYNTHETIC PEPTIDE
RHWRPKFRKFRWWRWHHGSC

SEQ ID NO: 47 - protein, 20 aa, Artificial Sequence
  SYNTHETIC PEPTIDE
FHKKRHFKRRHKFWWWRGSC

SEQ ID NO: 48 - protein, 20 aa, Artificial Sequence
  SYNTHETIC PEPTIDE
HKWHRKKFHKHRFKHHKGSC

SEQ ID NO: 49 - protein, 20 aa, Artificial Sequence
  SYNTHETIC PEPTIDE
WFWKHKKWRRHPRKWHWGSC

SEQ ID NO: 50 - protein, 20 aa, Artificial Sequence
  SYNTHETIC PEPTIDE
EPKLWFKPRRGGYRHRHGSC

SEQ ID NO: 51 - protein, 20 aa, Artificial Sequence
  SYNTHETIC PEPTIDE
HRKWHWPWKWPWKRKRWGSC

SEQ ID NO: 52 - protein, 20 aa, Artificial Sequence
  SYNTHETIC PEPTIDE
RPKPHRHWKPKFPHHHWGSC

SEQ ID NO: 53 - protein, 20 aa, Artificial Sequence
  SYNTHETIC PEPTIDE
WRRSTPVGPWTWFGKFLGSC

SEQ ID NO: 54 - protein, 20 aa, Artificial Sequence
  SYNTHETIC PEPTIDE
RRFRHWWRWRKRRRKWRGSC

SEQ ID NO: 55 - protein, 20 aa, Artificial Sequence
  SYNTHETIC PEPTIDE
EFSNPTAQVFPDFWMSDGSC